(19) United States
(12) Patent Application Publication
(10) Pub. No.: US 2017/0332992 A1
(43) Pub. Date: Nov. 23, 2017

(54) Miniaturized Phantoms for Quantitative Image Analysis and Quality Control

(71) Applicant: The Board of Trustees of the Leland Stanford Junior University, Stanford, CA (US)

(72) Inventors: Jan Liphardt, Stanford, CA (US); Debra M. Ikeda, Stanford, CA (US); Jafi A. Lipson, Stanford, CA (US); Weiva Sieh, Stanford, CA (US); Daniel L. Rubin, Stanford, CA (US); Ida Walworth, Stanford, CA (US); Jennifer S. Lee, Stanford, CA (US)

(73) Assignee: The Board of Trustees of the Leland Stanford Junior University, Stanford (CA)

(21) Appl. No.: 15/525,866

(22) PCT Filed: Nov. 12, 2015
(86) PCT No.: PCT/US2015/060462

§ 371 Date: May 10, 2017

Related U.S. Application Data
(60) Provisional application No. 62/079,438, filed on Nov. 13, 2014.

(51) Int. Cl. A61B 6/00; G06T 7/00

(52) U.S. Cl. CPC A61B 6/583; G06T 7/0012; A61B 6/4494; A61B 6/502; G06T 2207/30068

(57) ABSTRACT

Disclosed is a miniaturized phantom that can be placed against breast tissue during mammography. The phantom is provided with various radiological features that can be compared to the image of the breast tissue. The phantom is situated to be included in one or more mammography images. The phantom is at least partially opaque to the radiation of the image and contains features such as step wedges of different density, pillars that show radiation incidence, sweep gratings that show variations of radiation amplitude and a unique bar code to identify patients. The phantoms can be used in images containing them to assess various radiological features in a quantitative way.

### CROSS-REFERENCE TO RELATED APPLICATIONS

This application claims the benefit of U.S. Provisional Patent Application No. 62/079,438 filed Nov. 13, 2014, which application is incorporated herein by reference in its entirety.

### STATEMENT OF GOVERNMENTAL SUPPORT

This invention was made with Government support under contract CA143836 awarded by the National Institutes of Health. The Government has certain rights in the invention.

### BACKGROUND OF THE INVENTION

### Field of the Invention

The present invention relates to the fields of medical imaging and cancer risk assessment. In particular, the invention relates to the reproducible quantitative interpretation of high-resolution images of human tissues in terms of a patient's risk of future malignancy.

### Related Art

Presented below is background information on certain aspects of the present invention as they may relate to technical features referred to in the detailed description, but not necessarily described in detail. That is, individual compositions or methods used in the present invention may be described in greater detail in the publications and patents discussed below, which may provide further guidance to those skilled in the art for making or using certain aspects of the present invention as claimed. The discussion below should not be construed as an admission as to the relevance or the prior art effect of the patents or publications described.

### SPECIFIC PATENTS AND PUBLICATIONS

U.S. Pat. No. 5,844,965, “Method and apparatus for using film density measurements of a radiograph to monitor the reproducibility of x-ray exposure parameters of a mammography unit,” describes the use of a phantom for accreditation. The phantom used in the accreditation program consists of a plastic block and a wax insert that contains the artifacts. A radiograph of the insert by itself, that is, without the plastic block, is provided to the user to demonstrate the location of the artifacts. This image is also intended to demonstrate the maximum number of artifacts that can be visualized in a contact radiograph with essentially no excess scatter. In the accreditation program, the complete breast phantom is radiographed and the scatter helps to reduce the number of artifacts seen. Viewers score image quality on the basis of the number of artifacts seen.

U.S. Pat. No. 5,095,499, “Oriented mammography phantom,” describes a phantom with features that allow consistent placement on the detector array.

U.S. Pat. No. 4,655,716, “Contoured mammography phantom with skin,” disclosed a phantom that produces a more realistic x ray image of a breast and of its common anomalies such as masses, fibers and calcifications, so that an operator in training need not develop one set of mental images for the test object and another for actual breasts.

U.S. Pat. No. 7,667,191, “Deformable phantom apparatus,” discloses a deformable phantom apparatus for simulating motion of a patient's anatomy in 3D during breathing.

US 20120189175, “Method and system for analyzing tissue from images,” discloses a method of analyzing tissue from an image comprising providing an electronic image of tissue, determining a reference value from the image, establishing an hint representation of the image, and using the hint representation in analysis of the tissue to quantify the breast and compute a calibration error. Also disclosed is a system that runs an inner breast edge detection algorithm on the electronic image to detect the inner breast edge on the image, and refined the inner breast edge location if a calibration error is not acceptable. Also disclosed is automatic estimation of breast composition and temporal analysis of images.

US 20130272595, “Method for assessing breast density,” discloses methods of assessing breast density for breast cancer risk assessment applications. The methods include receiving digital image data (including FFDM and digitized film as well as other forms of imaging) including a plurality of pixels; calibrating the digital image data; performing a statistical analysis on the calibrated digital image data; and associating the statistically analyzed digital image data with a measure of risk for breast cancer.

Mammogram accreditation phantoms are known in the art for validating mammograms. An example is given at http(colon)(slash-slash)www(dot) cirsinc.com/products/all/47/mammographic-accreditation-phantomPdetails=specs. Known phantoms have fibers with diameters of 1.56, 1.12, 0.89, 0.75, 0.54, and 0.40 mm; specks with diameters of 0.54, 0.40, 0.32, 0.24, and 0.16 mm; and masses with decreasing diameters and thicknesses of 2.00, 1.00, 0.75, 0.50, and 0.25 mm (see Mammography Phantom Image Quality Evaluation (from the American College of Radiology 1999 Mammography Quality Control Manual). As discussed below, the present invention may also be used for quality control.

### BRIEF SUMMARY OF THE INVENTION

The following brief summary is not intended to include all features and aspects of the present invention, nor does it imply that the invention must include all features and aspects discussed in this summary.

The present invention comprises a device, a miniaturized calibration and quality control standard (e.g., a miniaturized mammography calibration standard) with particular internal architecture and composition, and associated mathematical and computational methods. The overall use of the device and associated methods is (1) to enable the quantitative interpretation of x-ray images (e.g., mammograms) in terms of patient-specific cancer risk and (2) to improve the early detection and classification of cancer, e.g., breast cancer. The present methods, using the presently disclosed phantom, further enable more definition of features obtained from an x-ray image (e.g., a mammogram) and obtain information about lesions or suspected masses.

The device and method can generate quantitative cancer risk by comparing numerical values obtained from one or more mammograms generated with the present phantom included in the image, by virtue of having been placed in contact with the breast tissue or next to the breast tissue during the imaging procedure. Quantitative values for image features, such as density, collagen features and the like are obtained and compared with reference values. The detection of an early increase or decrease of a quantitative feature can be used to better predict cancer risk and detect cancers such as breast cancers earlier. The present methods include a method for performing a mammogram, comprising: (a) placing a miniaturized phantom in contact with (or near to) a tissue (e.g., the breast) before imaging, (b) exposing the phantom and the tissue to radiation, and (c) obtaining an image (e.g., a mammogram) that includes the phantom and the subject's tissue (e.g., the breast), and (d) obtaining additional information from the phantom such as an estimate of the actual x-ray dose delivered to a specific patient during a specific procedure. The invention includes making and using a unique phantom that is configured to be in contact with (or near to) the breast during imaging. The phantom contains structural features that are imaged and can be used to detect and quantify features in the tissue image, such as density and anatomical features.

The present phantoms can be made of plastic and fabricated using 3-D printing, and incorporate additional materials such as paraffin, radio-opaque powders, and materials that change their properties when exposed to x-rays, such as unexposed x-ray film.

Aspects of the invention comprise a mammography phantom comprising one or more of (e.g., any combination of) a step wedge, a sweep grating, a distortion-measuring feature and an identification feature. In certain aspects, the step wedge comprises a series of adjacent sections of increasing predetermined vertical thicknesses; the sweep grating comprises parallel ribs with variable horizontal thickness and horizontal spacing; the distortion-measuring features comprise an array of vertical pillars of varying diameters; and the identification feature comprises an array of structures that create a bar code image in the mammogram.

Aspects of the invention include use of the present phantoms, including a method for preparing a mammogram, comprising: obtaining a mammogram image including a phantom in contact with a subject's breast during generation of the mammogram image, wherein the phantom comprises a structural feature selected from the group consisting of a step wedge, a sweep grating, a distortion-measuring feature, an identification feature, and any combination thereof.

Aspects of the invention include methods of x-ray imaging, e.g., performing a mammogram. The methods include obtaining an x-ray image (e.g., a mammogram) including a phantom in contact with a subject's tissue (e.g., a subject's breast when performing a mammogram) during generation of the x-ray image (e.g., a mammogram image), where the phantom includes one or more of the structural features described herein imaged during the x-ray imaging, e.g., during performing a mammogram.

Aspects of the invention further comprise a mammography phantom adapted and sized to be part of a mammogram image, comprising imaging structural features selected from the group consisting of a step wedge, a sweep grating, a distortion-measuring feature, an identification feature, and any combination thereof.

The present mammogram may further comprise one or more of the following structural features: a step wedge that comprises a series of adjacent sections of increasing predetermined vertical thicknesses; a sweep grating that comprises parallel ribs with variable thickness and spacing; a distortion feature that comprises an array of vertical pillars of variable diameters; and identification features that comprise an array of structures that creates a bar code image in the mammogram.

According to certain embodiments, the one or more structural features of the phantom include a step wedge. In certain aspects, the one or more features of the phantom include a sweep grating. According to certain embodiments, the one or more features of the phantom include pillars. In certain aspects, the one or more features of the phantom include both spatial and textural features. According to certain embodiments, the one or more features of the phantom include radiographic density features. In certain aspects, the one or more features of the phantom include an identification feature (e.g., a 1D, 2D or 3D barcode). The barcode may be different among different phantoms and can be used to uniquely identify a particular phantom with the barcode x-ray pattern in an x-ray image containing that phantom. According to certain embodiments, the present phantom also incorporates x-ray sensitive materials (such as x-ray film as used in a dosimeter badge) or electronic circuits (such as a MOSFET-based electronic dosimeter) that can be used to determine the actual x-ray dose delivered to a patient during a procedure. The phantom may include one or more of any of the features described above. For example, the phantom may include a step wedge, a sweep grating, pillars, an identification feature, a passive or active x-ray dose quantifier, and any combination thereof.

Also disclosed herein are phantoms comprising one or more x-ray imaging (e.g., mammography imaging, i.e. structural) features. According to certain embodiments, the one or more x-ray imaging features of the phantom include a step wedge. In certain aspects, the one or more x-ray imaging features of the phantom include a sweep grating. In certain aspects, the one or more x-ray imaging features of the phantom include pillars. According to certain embodiments, the one or more x-ray imaging features of the phantom include both spatial and textural features. In certain aspects, the one or more x-ray imaging features of the phantom include radiographic density features. According to certain embodiments, the one or more x-ray imaging features of the phantom include an identification feature (e.g., a 1D, 2D or 3D barcode). The phantom may include one or more of any of the x-ray imaging features described above. For example, the phantom may include a step wedge, a sweep grating, pillars, an identification feature, and any combination thereof. The phantom may be a mammography phantom that includes one or any combination of the x-ray imaging features described above.

Aspects of the present disclosure include collections of mammography phantoms. Phantoms of the collection include one or more mammography imaging features, which may be any of the imaging features described herein, in any desired combination. The mammography imaging features of members of the collection may be the same or different. In certain aspects, the mammography imaging features (e.g., one or any combination of a step wedge, a sweep grating, and identification feature, etc.) are varied between individual members in the collection to accommodate different breast tissue types. According to one embodiment, the mammography imaging features of members of the collection vary to accommodate different breast densities.

Also provided by the present disclosure are methods for analyzing an x-ray image (e.g., a mammogram) containing therein an image of a phantom and a tissue (e.g., breast tissue). The methods include normalizing pixel values in a tissue image with reference to an image of the phantom, and determining the resolution of the tissue by reference to known dimensions in the phantom. The methods further include measuring density of tissue (e.g., breast tissue) on a scale based on a phantom in the image and comparing that to a later image of the same tissue (e.g., the same breast tissue) and phantom. The methods further include analyzing an image relative to a specific phantom within the image to determine one or more of (i) extent of collagen alignment on spatial scales of microns to centimeters, (ii) the radial symmetry of spiculation around dense features, (iii) temporal changes of collagen alignment, and (iv) the magnitude of the local signal gradient at the boundary or regions with density changes.

### BACKGROUND OF THE INVENTION

One in 8 women will develop breast cancer during her lifetime, and 1 in 37 will die of this disease. Mammography is widely used to screen women for breast cancer, based on the clinical benefits of early detection. Over 38 million mammography procedures were reported in 2014.

Epidemiological studies have demonstrated that mammograms capture additional information beyond the presence or absence of breast cancer. For example, mammographic density is one of the strongest risk factors for breast cancer. Breast density refers to the amount of dense fibroglandular tissue visualized on a mammogram and this characteristic of the human breast has the highest attributable fraction of cancer risk, accounting for 16% of all breast cancers [1].

Despite the knowledge that mammographic density is strongly associated with breast cancer risk, mammography has been underutilized for risk stratification and prevention. Better risk stratification could help reduce costs, improve utilization of sensitive but costly modalities such as MRI, and increase the efficiency of screening programs by tailoring regimens according to each woman's risk.

The limited use of breast density in risk prediction models is in part due to paucity of robust quantitative measures of breast density. Clinically, breast density is routinely assessed using a qualitative categorical BI-RADS scale [2]: (a) almost entirely fatty; (b) scattered areas of fibroglandular density; (c) heterogeneously dense; and (d) extremely dense. In research, Cumulus [3] is widely used to obtain quantitative area-based measures of breast density on film screen mammograms. Both BI-RADS and Cumulus measures have subjective aspects and consequently vary substantially across readers.

Existing software packages seek to reduce variability and to provide semi-empirical metrics that can be used by clinicians to risk-stratify patients. For example, Hologic offers the Quantra™ Volumetric Breast Density Assessment tool. This software package estimates the volume of fibroglandular tissue and total breast volume, and reports the ratio of these values, the volumetric breast density, to the physician.

Recent developments in the basic sciences and in the clinic raise the possibility that there are other features of breast tissue, beyond area/volumetric breast density that are associated with cancer risk and influence cancer subtype and progression. These features include the extent of collagen alignment on spatial scales of microns to centimeters [4, 5], the radial symmetry of spiculation around dense features, temporal changes of collagen alignment, and the magnitude of the local signal gradient at the boundary of regions with density changes. As described in these associations are driving efforts to quantify them in patients or biopsy samples.

Although current mammography hardware and software solutions can provide area/volumetric breast density, the clinical workflow and the employed hardware and software are not currently optimized for quantification of microanatomical and spatial features as summarized in the preceding paragraph.

### BRIEF DESCRIPTION OF THE DRAWINGS

FIG. 1A-1F shows prototype designs and testing. FIG. 1A shows a first prototype: the image shows a 3D printed sweep grating embedded in molten paraffin and enclosed in a small plastic cylinder. Here the device consists of a series of about 15 parallel rib-like structures, having a progressive range of heights and inter rib distances. The coin provides a size scale showing that the device is less than a square inch in size. FIG. 1B shows two prototypes next to a standard phantom (front, see device from Gammex, Inc.) on a mammography unit. FIG. 1C shows a detail of the x-ray signal collected in those trials. FIG. 1D shows a top view schematic of a pre-production device. The device contains a compact array of features, namely a sweep grating 102 having elongated ribs, with an array of adjacent squares forming a step wedge 100, and an array of pillars 104 extending orthogonally to the ribs. A square barcode area 106 is fitted adjacent the pillars 104 and the step wedge 100. It contains a sweep grating with variable amplitude 102, elements such as a step wedge 100, elements for correcting distortions (five circular appearing pillars 104) and a x-ray visible 2D barcode 106 containing several regions of different radiodensity (or radiolucence), for unambiguous tracking of which specific phantom was used in a particular exposure. FIG. 1E shows a 3D printed version of design shown in FIG. 1D. FIG. 1F is an actual x-ray image of the present phantom. The image was generated using false color (not shown here), allowing the user, or an imaging software, to readily interpret results. In use, the x-ray image would be part of a tissue (breast) image. For convenience, the features are referred to as horizontal if in the plane of the image. In use, the phantom can be placed in any region of the tissue (breast) being imaged.

As is known in the art, 3D printers are commercially available and all start with making a virtual design of the object to be created. This virtual design is made in a CAD (Computer Aided Design) file using a 3D modeling program (for the creation of a totally new object) or with the use of a 3D scanner (to copy an existing object). A 3D scanner makes a 3D digital copy of an object. See for, for example, U.S. Pat. No. 7,766,641, U.S. Pat. No. 5,028,950, etc.

FIG. 2A-2D shows a mammogram of a woman with breast cancer with multiple lesions in a web of remodeled extracellular matrix. FIG. 2A shows 3D model system of breast cancer initiation and progression that recapitulates key aspects of human cancer (see for details reference [5], Shi, Q. M., et al., Rapid disorganization of mechanically interacting systems of mammary acini. Proceedings of the National Academy of Sciences of the United States of America, 2014. 111(2): p. 658-663), including the gradual formation of collagen patterns that mirror (1) the collagen tracts seen at the tumor/stromal boundary in primary breast tumors exhibiting increased propensity for metastasis and invasion and (2) the lines of radio-opaqueness seen in mammograms (compare FIG. 2A to FIG. 2C). The collagen patterns also mirror the TACS-3 (tumor-associated collagen signature 3) tracts that predict poor patient survival [4] (Conklin et al., Aligned collagen is a prognostic signature for survival in human breast carcinoma. Am J Pathol., 2011. 178(3) p. 1221-32). These findings are exploited here to demonstrate that breast tissue features, beyond simple area/volumetric measures of breast density, may increase cancer risk and can be evaluated using the present phantom. Potential phantom features include the extent of collagen alignment measured on spatial scales of microns to centimeters, the radial symmetry of spiculation around dense features, temporal changes of collagen alignment, and the magnitude of the local signal gradient at the boundary of regions with density changes. While these features can be quantified in biopsy, samples by optical imaging of tissue sections risk assessment would ideally use a routine noninvasive imaging modality such as mammography. FIG. 2B shows a collagen tract imaged adjacent to an acinus; FIG. 2C also shows a collagen tract, at a further detail; FIG. 2A shows vimentin and a collagen line. By comparing features shown in the mammogram with defined structures in the present phantom, previously unavailable data can be derived from the mammogram. As noted above, for example, characteristics of speculation in image can be analyzed in terms of size and density. See for details on spiculated lesions, Franquet al., “Spiculated lesions of the breast: mammographic-pathologic correlation,” Radiographics. 1993 July; 13(4):841-52.

Clinical imaging studies, mainly on older film-screen mammograms, suggest that localized textural features of parenchymal tissue are associated with breast cancer risk, independent of breast density.

Multi-scale Riesz filterbanks are used to characterize the morphological and textural properties of breast parenchyma in digital mammograms. Riesz wavelets quantify the local amount of directional image patterns at multiple scales, and are advantageous compared to other methods because they can exhaustively characterize image directions (steerable property) and scales (multiresolution). Textural features capturing the responses of the locally-steered texture models, combined with image pixel statistics, which encompass combinations of image scales and directions in regions of breast density, can predict cancer risk. Second-order Riesz wavelets are computed from the regions of breast density identified and segmented by Cumulus. The local morphological tissue properties of heterogeneities in dense breast tissue, arising from structural alterations related to underlying collagen structures in the breast that give rise to the breast density, are expressed as combinations of the responses of the oriented filters. The filters are used with multiple scales to analyze both fine morphological structures and coarser texture of breast anatomy. The present phantom can provide feature information useful in calculating Riesz features.

Further regarding texture features, Reference 4, described that aligned collagen is a prognostic signature for survival in human breast carcinoma; the present phantom can provide reference image features indicating size of associated collagen fibers and their radial alignment. For further example, Bredtfeld et al. propose the use of second harmonic optical imaging of tissue sections to assess risk [6].

### DETAILED DESCRIPTION

### Definitions

Unless defined otherwise, all technical and scientific terms used herein have the same meaning as commonly understood by those of ordinary skill in the art to which this invention belongs. Although any methods and materials similar or equivalent to those described herein can be used in the practice or testing of the present invention, the preferred methods and materials are described. Generally, nomenclatures utilized in connection with, and techniques of, cell and molecular biology and chemistry are those well-known and commonly used in the art. Certain experimental techniques, not specifically defined, are generally performed according to conventional methods well known in the art and as described in various general and more specific references that are cited and discussed throughout the present specification. For purposes of clarity, the following terms are defined below.

Ranges: For conciseness, any range set forth is intended to include any sub-range within the stated range, unless otherwise stated. As a non-limiting example, a range of 120 to 250 is intended to include a range of 120-121, 120-130, 200-225, 121-250 etc. The term “about” has its ordinary meaning of approximately and may be determined in context by experimental variability. In case of doubt, the term “about” means plus or minus 5% of a stated numerical value.

The term “phantom” refers, as is understood in the art, to a specially designed object that is scanned or imaged in the field of medical imaging to evaluate, analyze, and tune the performance of various imaging devices. A phantom is more readily available and provides more consistent results than the use of a living subject or cadaver, and likewise, in previous use, avoids subjecting a living subject to direct risk. Phantoms were originally employed for use in 2D x-ray based imaging techniques such as radiography or fluoroscopy, though more recently phantoms with desired imaging characteristics have been developed for 3D techniques such as MRI, CT, Ultrasound, PET, and other imaging methods or modalities.

The term “mammography” refers to using low-energy x-rays to examine the human breast, which is used as a diagnostic and screening tool. Included in the term “mammography” are numerous distinct technical implementations, differing in (1) detector technology (e.g., film or digital), (2) imaging dimension (e.g. 2D or 3D tomosynthesis), (3) the use of agents to increase local contrast (e.g., iodinated contrast agents), and (4) the number of energies used in the imaging (e.g., single energy, dual energy, or triple energy acquisitions). These variants of classical mammography are referred to with terms such as “digital mammography”, “Full Field Digital Mammography (FFDM)”, and “contrast-enhanced spectral mammography (CESM).” This progress is some years later than in general radiology. This relative delay is due to several factors: the higher spatial resolution demands of mammography, significantly increased expense of the equipment, concern by the FDA that digital mammography equipment demonstrate that it is at least as good as screen-film mammography at detecting breast cancers without increasing breast dose or the number of women recalled for further evaluation. The term mammogram accordingly refers to the x-ray image of the breast taken using these methods.

In describing the present miniaturized phantom, the term “vertical” may be used, for convenience, to refer to a feature that extends away from the plane of the phantom, wherein the phantom is adapted to be a flat or curvilinear surface to be comfortably placed against or adjacent the tissue, and “horizontal” then refers to an arrangement in parallel with a plane adapted to be placed against the tissue being imaged.

Overview

The present invention provides methods of quantitative interpretation of mammograms in terms of disease (e.g., cancer) risk. It overcomes barriers that include:

1. [Variation of Hardware]
Images collected on different mammography devices and manufacturers can be hard to compare quantitatively due to lack of cross-manufacturer standardization and lack of cross-manufacturer standards e.g., in the use of nonlinear and/or adaptive image thresholding.

2. [Variations in Image Acquisition Settings and Site-to-Site Differences in Workflow and Operator Training]
Images are collected using parameter settings (KV and mass) that vary among patients and even within the same patient on different imaging dates. Moreover, different clinical sites have different standard operating procedures, possibly leading to biases in e.g., typical degree of tissue compression and instrument settings.

3. [Variations in the Degree of Compression]
Compression of the breast is performed as a preparatory step to acquiring the images. The degree of compression varies within the same patient imaged on different dates. If there is less compression, the pixels in the image are generally darker due to thicker tissue being penetrated by the x-ray beam.

4. [Highly Complex and Variable Detector to Screen and Detector to File Transfer Functions]
The conversion of an x-ray attenuation measurement (e.g. a mammogram) into a digital image involves many steps, some of which locally modify the pixel intensities and scaling to accentuate certain image features at the expense of others. In general, the ability of x-ray hardware to accurately quantify features on multiple spatial scales is incompletely characterized, and it is not clear how to estimate actual hardware performance on a per-patient and per-image level.

5. [Changes in Signal Processing Algorithms]
Images collected on the same hardware can be hard to compare quantitatively over time due to changes of image processing algorithms.

6. [Mammography Phantoms are not Designed for Risk Prediction and Early Detection]
Contemporary mammography phantoms do not incorporate the patterns and textures that are most associated with risk and progression.

Reliable analysis and interpretation of imaging data requires the actual performance of the imaging hardware and software to be regularly quantified using an independent, defined standard. There are many potentially relevant performance characteristics of an imaging device, such as spatial resolution, pin-cushion distortion, or signal-to-noise ratios. Contemporary mammography phantoms are large, heavy, and thick. These phantoms are not designed to be placed directly next to the tissue during every exposure, and therefore, images do not typically contain defined spatial fiducials for subsequent quantitative interpretation. This further exacerbates barriers enumerated listed above.

It is expected that the present images will be processed and analyzed by computer means. The calculation methods described here can be applied to an image obtained with a breast area containing the specialized phantom described here. The image will be processed initially as a breast is x-rayed from top to bottom and from side to side. Typically, when a mammogram image is viewed, breast tissue appears white and opaque and fatty tissue appears darker and translucent. A digital mammogram may be obtained by known methods. In a digital mammogram, x-rays are still used. But they are turned into electric signals that can then be stored in a computer. This is similar to the way digital cameras take and store pictures. Thus, using the present invention, quantitative values of features (as described herein) may be stored and manipulated by software methods as described below.

### Miniaturized Mammography Phantom

It is demonstrated here that the barriers enumerated above can be addressed by placing a miniaturized mammography phantom next to the patient during mammography, such that each x-ray exposure and captured image contains both the sample (the tissue) and the present miniaturized phantom. The phantom may be in direct contact with the breast during the procedure, which may involve squeezing the breast against the phantom.

Such a mammography phantom is different from the mammography phantoms currently in practice, which are imaged periodically as part of mammography quality assurance programs to ensure images are uniform and the mammography setting produce image density expected. These phantoms are imaged without the patient. On the other hand, the present invention comprises use of a phantom that is imaged with the patient, providing a standard for calibrating or normalizing the image pixel values and assessing multiple other parameters of the imaging system for every exposure and for every patient. The phantom may actually be compressed against breast tissue during a mammography procedure.

Moreover, the present miniaturized mammography phantom ideally contains examples of the specific spatial and textural features that are highly associated with risk and progression, rather than only containing generic features such as a step wedge or a 1 cm diameter sphere. Conceptually, the reader may imagine trying to reliably find a specific shape (e.g., a rocket) in an image (e.g., from a satellite camera). If the imaging system injected that specific shape at a specific “control” location in every image, then subsequent algorithms/analysis could be benchmarked and optimized on an exposure-by-exposure and image-by-image basis, improving detection probability and allowing estimation of the actual false positive and false negative likelihood.

By synthesizing these points, we present device illustrate that there is a fundamental connection between a patient's features and a mammography phantom. For example, patients with dense tissue will likely benefit from a phantom with comparable (high) density and further containing the specific risk predictors most relevant to that specific patient or patient cohort (defined by e.g., age, weight, previous history of breast cancer, currently in treatment for breast cancer using aromatase inhibitors).

Upon consideration of the above list, the miniaturized mammography phantoms can be produced by rapid fabrication methods such as 3D printing of FDA-compliant materials such as ABS plastics and UV curable acrylics, with and without incorporation of additional materials such as waxes, powders, and chemicals such as barium sulfate, bismuth subcarbonate, bismuth oxychloride, bismuth trioxide, and tungsten. The powdered material may be applied in a predefined thickness either instead of 3D printing or in addition to 3D printing. The powdered material can be used to form one or more of the structural features described.

A further structural feature that may be included in the phantom is an x-ray sensitive material. In this case, the intensity of the radiation being received by the phantom (and the adjacent tissue) will produce a corresponding change in the x-ray sensitive material. The x-ray sensitive material may be, for example, such as x-ray film as used in in a dosimeter badge, or a miniature electronic circuits (such as a MOSFET-based electronic dosimeter) that can be used to determine the actual x-ray dose delivered to a patient during a procedure.

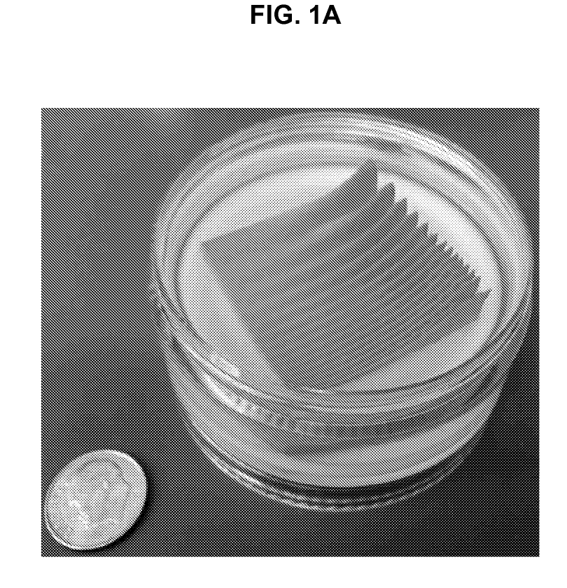

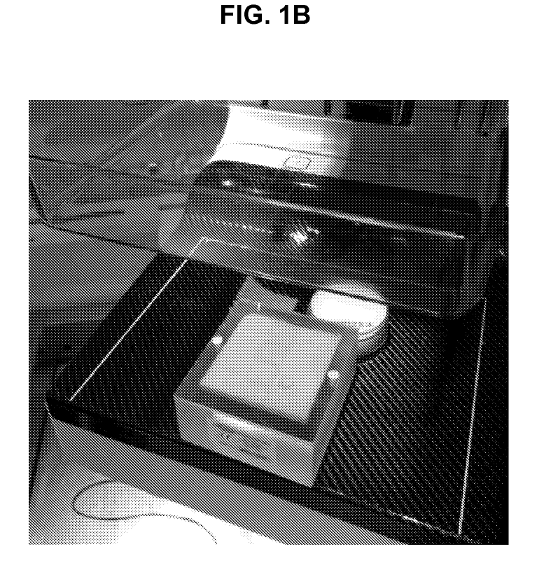

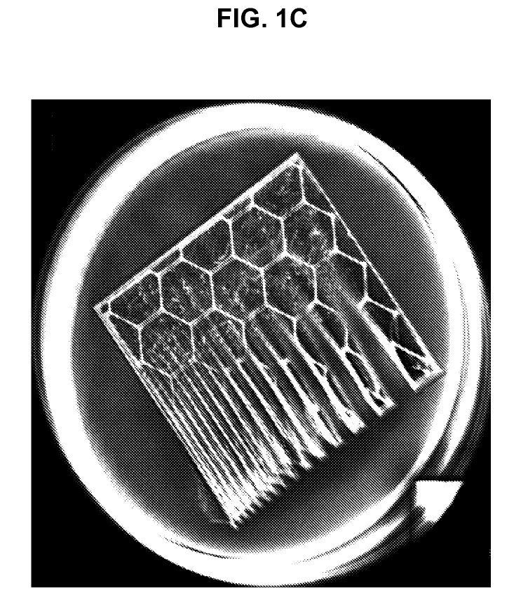

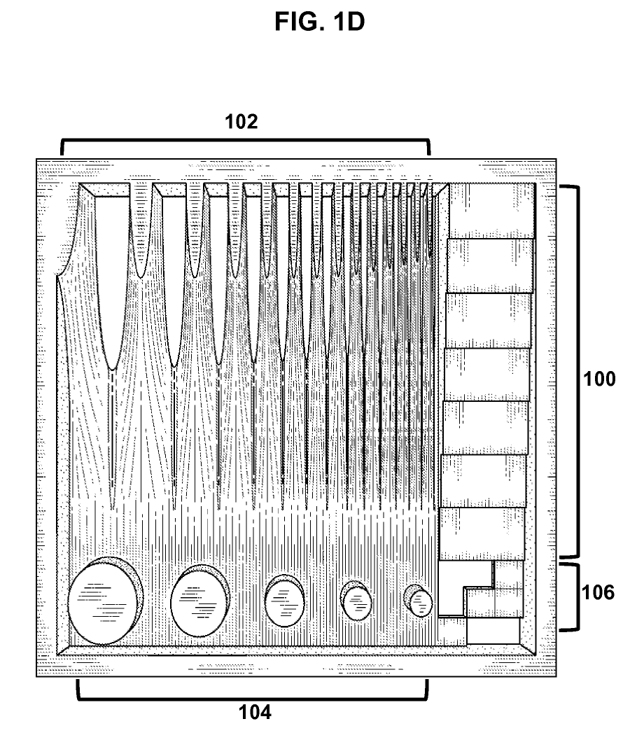

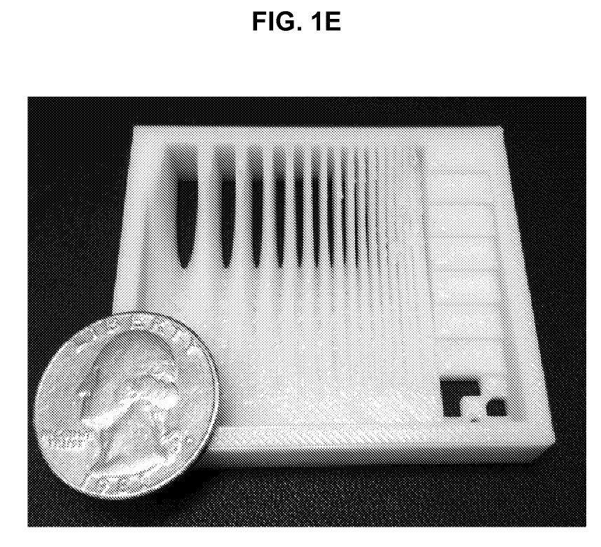

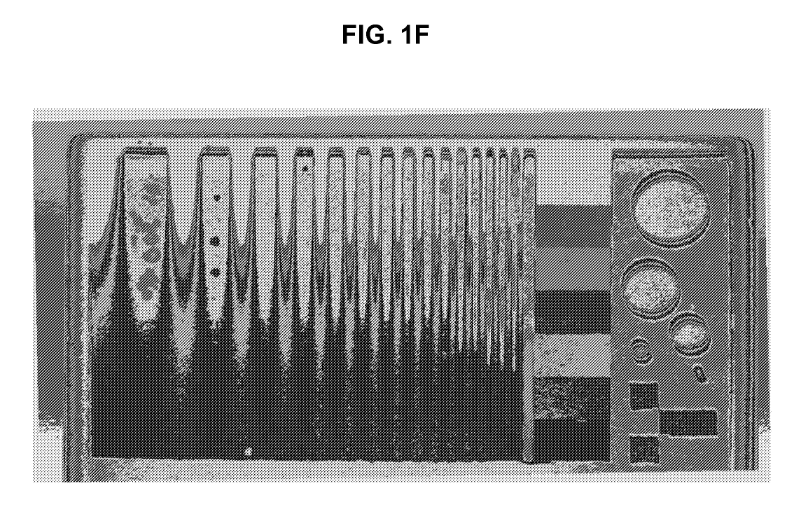

Thus, the methods and products described here embody a miniaturized mammography phantom with design characteristics and composition that allow the phantom to be placed next to a tissue during imaging, such that each x-ray exposure and captured image contains both the sample (the tissue) and the calibration standard/phantom (FIG. 1B). Such design characteristics include:
(1.1) height smaller than 30 mm,
(1.2) width and length smaller than 150 mm, and
(1.3) an aspect ratio that minimizes impingement of the phantom on the space available for the tissue and image markup.

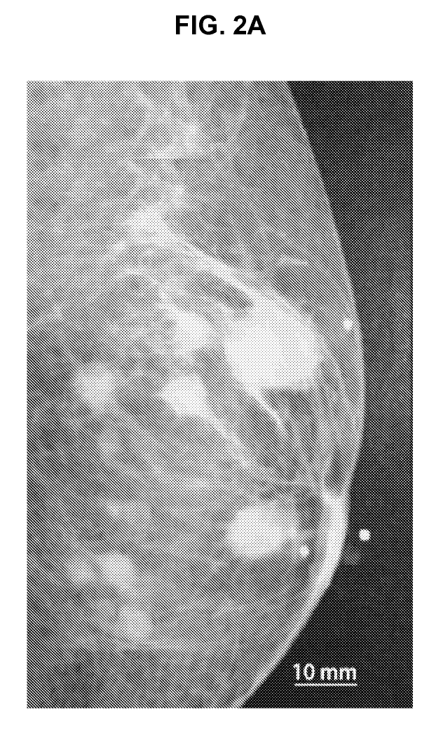

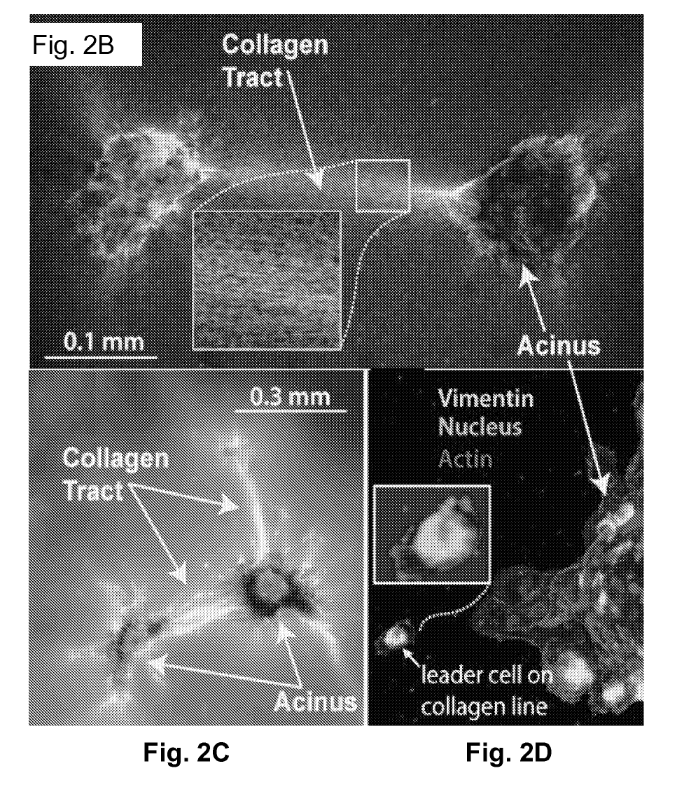

In a first embodiment, the miniaturized mammography phantom incorporates standard features such as a step wedge (FIG. 1D, ‘step wedge’ 100) to allow measurement of the linearity and the dynamic range of the detector/software combination and quantitative comparison with conventional phantoms, such as the mammography accreditation phantom. A step wedge provides a known linear progression of x-ray attenuation. By comparing the measured intensity changes in the region of the step wedge to the known x-ray attenuation of the step wedge, the linearity and the dynamic range of the detector/software combination can be determined. Knowledge of the linearity and the dynamic range of the measurement system are critical e.g., for quantitative risk prediction. For example, the probability and extent of mammary disorganization are in part controlled by the mechanical compliance of the environment surrounding the mammary acini, as shown in FIG. 2C of reference [4] for elastic moduli of 150 to >5000 Pa. Critically, mammary acini do not respond to substrate compliance in an all-or-nothing (binary) manner, but exhibit a graded response, with higher compliances resulting in more extensive disruption and pre-malignant signaling [4]. The mechanics of a tissue, in turn, are influenced by collagen concentration, one of several components of overall radiographic contrast. Therefore, quantitative risk prediction based on connections such as reported in [4] require knowledge of the linearity and the dynamic range of the detector/software combination, since otherwise the risk models would entirely fail or underperform, by e.g., under- or over-predicting risk.

In a further embodiment, the miniaturized mammography phantom incorporates internal structures and features that emulate specific spatial and textural signatures of at-risk tissue. For example, a variable amplitude sweep grating may contain a grating, e.g., of the parametric form z<3.0+Sin [Exp[0.034*x]*0.4*x]*0.03*Exp[0.138*y]. A sweep grating (e.g., FIG. 1D, ‘sweep grating’ 102) can be used to estimate the actual transfer function of the instrument on the spatial feature scales most relevant to quantitative assessment of tissue microanatomy and risk. Beyond scalar parameters of a tissue, such as collagen concentration and elastic modulus, as discussed in the preceding paragraph, it is now increasingly understood that vectorial (directional) features of anatomy are connected to disease risk and associated with cancer stage. Relevant directional collagen microanatomical features are variously denoted as fibers, tracts, cables, straps, or lines. In general, these features represent regions of highly aligned collagen. These collagen features have been observed in systems ranging from single cells and tumor explants to human clinical samples. Regions of aligned collagen that extend radially from the tumor/stromal boundary in the human breast are associated with poor outcomes [4, 6]. In vitro model systems are beginning to provide specific data on which collagen features, on which spatial scales, increase cancer risk. We have found in 3D model systems that mammary acini seeded onto substrates with collagen lines disorganize more readily than when the acini are placed on unaligned collagen. These collagen lines have typical lateral dimensions of −0.1 mm and can extend over distances of more than 1 mm. Quantitative risk prediction based on spatial aspects of tissue microanatomy (e.g., such as reported in [1, 2, 4, 6]) requires knowledge of how well the imaging device (the detector/software combination) detects and reproduces those specific features. The sweep grating design was chosen to incorporate spatial frequencies from 0.4 lines per mm to 3 lines per mm, covering major features of collagen microanatomy. By placing the sweep grating next to the patient's tissue and simultaneously imaging both the sweep grating and the tissue, each exposure can be (1) validated and (2) assessed for instrument-specific distortions of the spatial content of the image. For example, an image in which sweep grating lines 15 and 16 are blurred together would indicate poor spatial resolution of risk and disease relevant microanatomical features, suggesting reacquisition and/or further studies.

In a further embodiment, the miniaturized mammography phantom incorporates internal structures and features that emulate specific spatial and textural signatures of tumor progression. For example, a 3 mm diameter ABS sphere with a bundle of thin fibers with diameter 0.1 mm and length 1 mm extending radially outwards can be used to estimate the actual transfer function of the instrument on the spatial feature scales most relevant to detection of tumors that have just begun to breach the basement membrane and engage stromal collagen 1, denoting invasion.

In a further embodiment, the miniaturized mammography phantom incorporates internal structures and features that allow specific optical aberrations and distortions to be measured for each exposure. For example, a series of vertical pillars with variable diameters of 0.5 to 5 mm (FIG. 1D, ‘pillars’ 104) can be used to measure the pincushion distortion of the instrument. Knowledge of the pincushion distortion of the instrument is important for risk prediction since it sets a fundamental limit on the extent to which risk-related features can be assessed in an image.

In a further embodiment, the miniaturized mammography phantom incorporates internal identification structures and features that allow each specific phantom to be unambiguously and permanently identified by simple inspection of the x-ray image without recourse to file headers or manual annotation of the patient's medical record. This can be achieved with an internal x-ray visible 2D barcode (FIG. 1D, ‘barcode’ 106).

In a further embodiment, the miniaturized mammography phantom incorporates two or more of the internal features described in the preceding paragraph.

In a further embodiment, the miniaturized mammography phantom is permanently or semi-permanently incorporated within the mammography instrument, between the x-ray source and the detector, e.g., through a slot, clip, or internal drawer mechanism.

In a further embodiment, the miniaturized mammography phantom is composed of materials that also provide contrast in other imaging modalities, such as in magnetic resonance imaging, positron emission tomography, or CT, a form of x-ray imaging that uses higher x-ray energies compared to mammography. A miniaturized multimodal phantom is useful for providing calibration and registration data for integration of two or more imaging modalities.

In a further embodiment, the miniaturized mammography phantom is disposable and used only for a limited time and/or number of exposures. A disposable or limited-exposure phantom addresses concerns relating to gradual temporal degradation of the phantom e.g., due to x-ray exposure or heat-sterilization.

In a further embodiment, the phantom incorporates x-ray sensitive materials (such as x-ray film as used in in a dosimeter badge) or electronic circuits (such as a MOSFET-based electronic dosimeter) that can be used to determine the actual x-ray dose delivered to a specific patient during a specific procedure.

In a further embodiment, the miniaturized mammography phantom is personalized based on clinical characteristics of a particular patient and/or a specific patient subpopulations. For example, patients with denser-than average tissue will benefit from phantoms with comparable (high) density, allowing quantification of performance characteristics of the hardware and software that are most relevant to a particular patient.

### Examples of Clinical Use of the Miniaturized Phantom

1. Pixel Value Normalization.

In this application, the phantom will be placed in proximity to the patient during imaging, such that each electronic image contains an image of the tissue and an image of the miniaturized phantom. Since the architecture and composition of the phantom are known, the signal from the tissue can be normalized to the phantom. The purpose of normalizing each separate exposure to an absolute standard is to allow quantitative comparison of images taken with different hardware and hardware settings, taken at distinct clinical sites at different times, and by different x-ray technicians. Such normalization and quantification is currently not possible because the relationship between the tissue (and its x-ray absorption characteristics) and the values of the image pixels recorded by the instruments varies. If it were possible to calibrate the pixels within each mammography image to an absolute standard (the basis of our invention), then the absolute values of these pixels could be more reliably be used in methods such as cancer risk models that consider these values as inputs.

2. Estimation of Actual Detection Probability on a Per Image and Per-Patient Basis.

A critical aspect of any clinical measurement is estimation of the false-positive and false-negative rates of a particular measurement done on a particular patient. For example, if cancer risk is associated with collagen tracts with a width of 200 microns, then an image with a lower effective resolution—for whatever reason such as poor collimation—will be fundamentally unable to detect that feature even if it is present. From a clinical perspective, this particular measurement has thus failed, since it is non-informative, and the ideal clinical outcome would be to alert the clinician so that additional measurements can be performed on that specific patient. This is especially critical since the effective resolution of an imaging system is not a static property of the imaging system, but can change over time and can depend on the characteristics of the sample (e.g., thickness) and on where the sample has been placed relative to the x-ray source and detector.

In this application, the phantom is placed in proximity to the patient during imaging, such that each electronic image contains an image of the tissue and an image of the miniaturized phantom. Since the composition and the specific lateral and vertical dimensions of all features within the phantom are known, the person or computer analyzing the image can readily determine whether a particular image passes a minimal quality/resolution acceptance threshold. For instance, if the smallest pillar (FIG. 1D, smallest of pillars 104) in the phantom cannot be well discriminated, the effective resolution or that particular exposure is poorer than the dimensions of the pillar, which—depending on the application—may suggests a re-exam with the same or another imaging modality.

3. Facilitate Early Detection of Breast Cancer.

A key indicator of malignancy is local (mm- or cm-scale) changes of breast density over time. Such quantitative temporal comparison is greatly facilitated by normalization of the signal to an absolute standard that is guaranteed not to change over time (e.g., the present phantom). The problem is especially acute if a women changes healthcare providers or moves from one continent to another. In this case, regional differences in procedures, training, hardware, and software can introduce image-to-image variations that swamp or obscure early indicators of malignancy.

In this application, as in applications 1 and 2, the phantom is placed in proximity to the patient during imaging, such that each electronic image contains an image of the tissue and an image of the miniaturized phantom. Since the composition and the internal architecture of the phantom are known, all images can be normalized relative to the phantom, enabling absolute quantification of images to one-another.

4. Facilitate Automatic Assessment of Mammographic Density and Tissue Composition.

Clinically, breast density is routinely assessed using a qualitative categorical BI-RADS scale [2]: (a) almost entirely fatty; (b) scattered areas of fibroglandular density; (c) heterogeneously dense; and (d) extremely dense. In research, Cumulus [3] is widely used to obtain quantitative area-based measures of breast density on film screen mammograms. Both BI-RADS and Cumulus measures have subjective aspects and consequently vary substantially across readers. Existing software packages seek to reduce variability and to provide semi-empirical metrics that can be used by clinicians to risk-stratify patients. For example, Hologic offers the Quantra™ Volumetric Breast Density Assessment tool. This software package estimates the volume of fibroglandular tissue and total breast volume, and reports the ratio of these values, the volumetric breast density, to the physician.

In this application, as in applications 1-3, the phantom is placed in proximity to the patient during imaging, such that each electronic image contains an image of the tissue and an image of the miniaturized phantom. Since the composition and the internal architecture of the phantom are known, all images can be normalized relative to the phantom, enabling assessment of mammographic density relative to an absolute standard. Assessment and calculation of “absolute” mammographic density will entail consideration of the actual thickness of the compressed breast, the degree of compression of the breast, and the particular tissue composition of the breast.

5. Facilitate Estimation of Breast Cancer Risk.

Recent developments in the basic sciences and in the clinic raise the possibility that there are other features of breast tissue, beyond area/volumetric breast density that are associated with cancer risk and influence cancer subtype and progression. These features include the (1) extent of collagen alignment on spatial scales of microns to centimeters [4, 5], the (2) radial symmetry of spiculation around dense features, (3) temporal changes of collagen alignment, and the (4) magnitude of the local signal gradient at the boundary of regions with density changes. As discussed below, these four variables can be quantified using the present phantom and imaging methods. These associations are driving efforts to accurately quantify them in patients or biopsy samples. For example, Bredtfeld et al. propose the use of second harmonic optical imaging of tissue sections to quantify so-called “tumor associated collagen signatures” for human breast carcinoma prognosis [6]. Although current mammography hardware and software solutions can provide area/volumetric breast density, the current clinical workflow and the employed hardware and software are not currently optimized for quantification of microanatomical and spatial features as summarized in the preceding paragraph. The overall goal of this application is to improve early detection and provide high-quality patient-specific risk estimates.

In this application, as in applications 1-4, the phantom is placed in proximity to the patient during imaging, such that each electronic image contains an image of the tissue and an image of the miniaturized phantom. Since the composition and the internal architecture of the phantom are known, the phantom can be used for at least 3 different but complementary purposes.

(1) to correct optical distortions (such as pincushion distortions) that impair ability to quantify risk and disease-relevant image features as e.g., reported by Bredtfeld et al. [6] such as fiber curvature, width, length, alignment, and the proximity and relative angle of the fibers to other anatomical structures which are visible in mammography e.g., as mammographically dense regions.

(2) to normalize images taken at different sites and at different times to an absolute standard, allowing quantitative assessment and better detection of small local density changes and other anatomical alterations.

(3) to establish formal detection thresholds, detection probabilities, and risk-assessment quality, on a per image and per patient basis.

The present methods also provide algorithms and methods for the following mammogram processes:
1. correcting 2D and 3D imaging distortions by reference to the known standard present within each x-ray exposure;
2. quantifying specific internal structures and features of tissue microanatomy; and
3. providing patient-specific risk estimates based on (2)

Mathematical and computational algorithms according to the present invention are (1) designing patient-personalized phantoms for risk-assessment and cancer detection and (2) using the information provided by the phantom to calibrate, correct, and facilitate the interpretation of the mammogram.
4. Providing standardized measurements of breast density (whiteness) and breast thickness

### REFERENCES

1. Boyd, N. F., et al., Mammographic density and the risk and detection of breast cancer. N Engl J Med, 2007. 356(3): p. 227-36.
2. Mercado, C. L., BI-RADS update. Radiol Clin North Am, 2014. 52(3): p. 481-7.
3. Byng, J. W., et al., The quantitative analysis of mammographic densities. Phys Med Biol, 1994. 39(10): p. 1629-38.
4. Conklin, M. W., et al., Aligned Collagen Is a Prognostic Signature for Survival in Human Breast Carcinoma. American Journal of Pathology, 2011. 178(3): p. 1221-1232.
5. Shi, Q., et al., Rapid disorganization of mechanically interacting systems of mammary acini. Proc Natl Acad Sci USA, 2014. 111(2): p. 658-63.
6. Bredfeldt, J. S., et al., Automated quantification of aligned collagen for human breast carcinoma prognosis. J Pathol Inform, 2014. 5: p. 28.

### CONCLUSION

The above specific description is meant to exemplify and illustrate the invention and should not be seen as limiting the scope of the invention, which is defined by the literal and equivalent scope of the appended claims. Any patents or publications mentioned in this specification are intended to convey details of methods and materials useful in carrying out certain aspects of the invention which may not be explicitly set out but which would be understood by workers in the field. Such patents or publications are hereby incorporated by reference to the same extent as if each was specifically and individually incorporated by reference and contained herein, as needed for the purpose of describing and enabling the method or material referred to.

What is claimed is:

1. A mammography phantom adapted and sized to be part of a mammogram image, comprising a structural feature selected from the group consisting of: a step wedge, a sweep grating, a distortion-measuring feature, an identification feature, and any combination thereof.

2. The mammography phantom of claim 1, wherein the step wedge comprises a series of adjacent sections of increasing predetermined vertical thicknesses.

3. The mammography phantom of claim 1 or 2, wherein the sweep grating comprises parallel ribs with variable horizontal thickness and horizontal spacing.

4. The mammography phantom of any one of claims 1 to 3, wherein the distortion-measuring features comprise an array of vertical pillars of varying diameters.

5. The mammography phantom of any one of claims 1 to 4, wherein the identification feature comprises an array of structures on the phantom that create a bar code image in the mammogram.

6. A mammography phantom of claim 1 comprising a step wedge, a sweep grating, a distortion-measuring feature and an identification feature, wherein:
(a) the step wedge comprises a series of adjacent sections of increasing predetermined vertical thicknesses;
(b) the sweep grating comprises parallel ribs with variable horizontal thickness and horizontal spacing;
(c) the distortion-measuring features comprise an array of vertical pillars of varying diameters; and
(d) the identification feature comprise an array of structures that create a bar code image in the mammogram.

7. A mammography phantom according to any one of claims 1 to 6, further comprising a structural feature formed of a defined layer of a powdered radiographic material applied to the phantom.

8. The mammography phantom of claim 7 wherein the powdered radiographic material is one of barium sulfate, bismuth subcarbonate, bismuth oxychloride, bismuth trioxide, and tungsten.

9. A mammography phantom according to any one of claims 1 to 8, further comprising an x-ray sensitive material, wherein the material is exposed to an x-ray when the phantom is in use.

10. A collection of mammography phantoms according to any one of claims 1 to 9, wherein the structural features are varied between individual members in the collection to accommodate different breast tissue types.

11. The collection according to claim 10, wherein the different breast tissue types differ according to breast density.

12. A method for preparing a mammogram, comprising:
obtaining a mammogram image including a phantom in contact with a subject's breast during generation of the mammogram image, wherein the phantom comprises a structural feature selected from the group consisting of: a step wedge, a sweep grating, a distortion-measuring feature, an identification feature, and any combination thereof.

13. The method according to claim 12, wherein the step wedge comprises a series of adjacent sections of increasing predetermined vertical thicknesses.

14. The method according to claim 12, wherein the sweep grating comprises parallel ribs with variable horizontal thickness and horizontal spacing.

15. The method according to claim 12, wherein the distortion-measuring features comprise an array of vertical pillars of varying diameters.

16. The method according to claim 12, wherein the identification feature comprises an array of structures on the phantom that create a bar code image in the mammogram.

17. The method according to one of claim 12 wherein the phantom comprises a step wedge, a sweep grating, a distortion-measuring feature, and an identification feature, wherein the step wedge comprises a series of adjacent sections of increasing predetermined vertical thicknesses; the sweep grating comprises parallel ribs with variable horizontal thickness and horizontal spacing; the distortion-measuring features comprise an array of vertical pillars of varying diameters; and the identification feature comprise an array of structures that create a bar code image in the mammogram.

18. A method for analyzing a mammogram containing therein a phantom and breast tissue, comprising one or more steps of:
a. normalizing pixel values in a tissue image with reference to an image of the phantom;
b. determining the resolution of the tissue by reference to known dimensions in the phantom;
c. measuring density of breast tissue on a scale based on a phantom in the image and comparing that to a later image of the same breast tissue and phantom;
d. measuring actual thickness of the compressed breast, the degree of compression of the breast, and the particular tissue composition of the breast using a phantom placed in proximity to the breast tissue in each image; and
e. analyzing an image relative to a specific phantom within the image to determine one or more of (i) extent of collagen alignment on spatial scales of microns to centimeters, (ii) the radial symmetry of spiculation around dense features, (iii) temporal changes of collagen alignment, and (iv) the magnitude of the local signal gradient at the boundary or regions with density changes.

19. The method of claim 18 wherein the phantom is adapted and sized to be part of a mammogram image and comprises an imaging feature selected from the group consisting of: a step wedge, a sweep grating, a distortion-measuring feature, an identification feature, and any combination thereof.

20. The method of claim 18, wherein the step wedge comprises a series of adjacent sections of increasing predetermined vertical thicknesses.

21. The method of claim 18, wherein the sweep grating comprises parallel ribs with variable thickness and spacing.

22. The method of claim 18, wherein the distortion features comprise an array of vertical pillars of variable diameters.

23. The method of claim 18, wherein the identification features comprise an array of structures that creates a bar code image in the mammogram.

24. The mammogram phantom of claim 1, the method of claim 12, or the method of claim 18, wherein the phantom consists of plastic material formed on a 3-D printer.
